(19) United States
(12) Patent Application Publication
(10) Pub. No.: US 2014/0315290 A1
(43) Pub. Date: Oct. 23, 2014

(54) LOW-COST PHOTOBIOREACTOR

(71) Applicant: INTERNATIONAL LTD., Richmond (CA)

(72) Inventor: Soheyl Mottahedeh, Coquitlam (CA)

(73) Assignee: INTERNATIONAL LTD., Richmond, BC (CA)

(21) Appl. No.: 14/362,904

(22) PCT Filed: Oct. 22, 2012
(86) PCT No.: PCT/CA2012/050750

§ 371 Date: Jun. 4, 2014

(30) Foreign Application Priority Data
Dec. 7, 2011 (CA) 2,761,251
Jan. 16, 2012 (CA) 2,764,291

(51) Int. Cl. C12M 1/00; C12M 3/06; C12M 1/12; C12M 3/00; C12M 1/42; C12M 1/34

(52) U.S. Cl. CPC C12M 21/02; C12M 35/02; C12M 41/00; C12M 23/04; C12M 23/46; C12M 29/20; C12M 31/00; C12M 31/10; C12M 27/16; C12M 23/22; C12M 23/26
USPC 435/288.7; 435/292.1; 435/287.1

(57) ABSTRACT

The present invention provides a photobioreactor comprising at least one translucent flexible sheet shapable by a support assembly forming thereby an elongated channel adapted for biomass production therewithin. Kits for making a photobioreactor and a floatable photobioreactor are also provided.

### FIELD OF THE INVENTION

The present invention relates to a bioreactor for production of biomass and more particularly to a low-cost, high surface-to-volume trough-like elevated pond that integrates features of photobioreactors such as transparency/translucency from all directions, closed environment, efficiency in the mixing of gases and temperature control. The configuration, fast erection, collapsibility and temperature control of the device may also apply to troughs for animal feed, to fish and shrimp culture and to fast erection of mini-greenhouses for agricultural purposes.

### BACKGROUND

The current energy crisis has prompted interest in alternative energy, bringing a great deal of attention to the production of algae biofuels. Beyond biofuels, commercial algae farming is also important to medicine, food, chemicals, aquaculture and production of feedstocks. One major obstacle to the production of biofuels is the commercial scale-up for mass culture, temperature control of algae and the high cost associated with such a culture.

The vast number of different bioreactor concepts is testimony that the best algal farming bioreactors are still to be found. Most bioreactor designs are not suitable for commercial use due to cost and scale-up problems. In contrast with bioreactors, pond technologies are commercially viable today, but have well-established problems of their own. Integrated technologies might provide the control offered through closed bioreactors and the scalability afforded by open ponds.

To appreciate the value of attempts made and of associated prior art, a short review of recent studies and related publications is presented:

According to Mario R. Tredici: “Outdoors, under full sunlight, the photosynthetic efficiency drops to one tenth-one fifth of the values observed at low irradiances. The major causes for this inefficiency are the light saturation effect (LSE) and photoinhibition, phenomena that strongly limit the growth of microalgae in outdoor culture, although these because of the high cell density, are light-limited. The main problem is that photosynthetic apparatus of phototrophs saturates at low irradiances and that, at irradiances above saturation, the absorbed photons are used inefficiently and may cause cell injury. Several strategies to overcome the LSE and photoinhibition have been proposed, based on engineering (light dilution, ultra high cell density culture, high turbulence), physiologic (photoacclimation, nutrient deprivation) or genetic.” (Tredici M. R. (2004) Mass production of microalgae: photobioreactors. In Richmond A (ed.), Handbook of Microalgae Culture. Blackwell Publishing, Oxford (UK), pp 178-214.

Dimanshteyn taught in U.S. Pat. No. 7,824,904 that photobioreactors generally consist of a container containing a liquid growth medium that is exposed to a light source. However, the configuration of the photobioreactor often prevents the light from penetrating more than a few centimeters from the surface of the liquid. This problem reduces the efficiency of the photobioreactor, and was recognized in “Solar Lightning for Growth of Algae in a Photobioreactor” published by the Oak Ridge National Lab and Ohio University. Light delivery and distribution is the principle obstacle to using commercial-scale photobioreactors for algae production. In horizontal cultivator systems, light penetrates the suspension only to 5 cm leaving most of the algae in darkness.

As described in Healthy Algae, Fraunhofer Magazine, January 2002, algae are a very undemanding life form—they only need water, CO₂, nutrients and sunlight. However, providing sufficient sunlight can be a problem in large scale facilities. As the algae at the surface absorb the light, it does not penetrate to a depth of more than a few millimeters. The organism inside the unit gets no light and cannot grow, explains Walter Troesch, who has been cultivating algae for years. One of the problems with growing algae in any kind of pond is that only in the top 1-4 or so of the pond receives sufficient solar radiation for the algae to grow. In effect, this means that the ability of a pond to grow algae is limited by its surface area, not by its volume.

In summary, the ability of a pond to grow algae is limited by its surface area, not by its volume. Therefore limitations in prior documents are examined in consideration of the above findings.

Traditional procedures employed for culturing autotrophic organisms have involved the use of shallow open ponds or open channels exposed to sunlight. Not surprisingly this comparatively crude method has proved impracticable for production of pure high grade products because of such problems as invasion by hostile species (sometimes producing dangerous toxins), other pollution (such as dust), difficulty in the control of such variables as nutrient ratios, temperature and pH, intrinsically low yield because of escape of carbon dioxide to the atmosphere and inefficient use of light to illuminate only the top portion of the biomass.

Somewhat more sophisticated attempts have involved the use of horizontally disposed large diameter transparent plastics tubes for biomass production. The problems of such a system include the low density of biomass in the liquid within the tubes, coating of the pipes by algae due to low velocity flow passing through, thus reducing transparency, overheating in summer weather, high land usage and high energy input to displace large amount of over diluted water.

Now, looking closely at receptacles disclosed in prior documents and more particularly for potential use as low-cost raceway-type pond or photo bioreactor, a number of inventions are examined.

U.S. Pat. No. 7,069,875 to Warecki (“Warecki”) discloses a large and low cost portable raceway or vessel for holding flowable materials. The vessel has a body formed of an elongate rollable sheet of buoyant material that, when assembled into an upwardly concave vessel has bulkheads at its ends to give it its half-rounded shape. The large vessel is self-supporting in both water and land. The Warecki vessel suffers from a number of limitations. Joining of parts such as bulkheads to the body of the vessel requires welding, chemical bonding, and- or mechanical fastening. Also, to maintain the shape of the pond, bulkhead bow frames must be positioned inside the vessel, dividing the space into closed compartments that are fastened mechanically or chemically to the body, although some unsecured movable compartments are used. Also, no provision of thermal control is provided.

WO2011016735 to Dalrymple discloses an erectable trough for animal feed. The plastic sheet disclosed by Darlymple is bent into a U-shaped trough with opposite side walls being supported upright by tension wires through perforations in the side walls. As disclosed, the trough is not waterproof and not suitable for a closed trough-like pond.

U.S. Pat. No. 5,846,816 to Forth (“Forth”) discloses a biomass production apparatus including a transparent chamber which has an inverted, triangular cross-section. Although the “Forth” bioreactor promotes the growth of biological matter, it contradicts the principles extensively tested by Tredici, Fraunhofer and National Labs that assert the need to maximize exposed surface area to sunlight relative to the volume displaced. Furthermore, the disclosed chamber is expensive to manufacture. Finally, the constant circulation of the liquid required by “Forth” interferes with the growth of some types of biological matter. For instance, fully differentiated aquatic plants from the lemnaceae or “duckweed” family are fresh-water plants that grow best on the surface of the water. Such surface growing plants typically prefer relatively still water to support and promote optimal growth.

Often, the importance of the surface area directly exposed to sunlight and which can benefit from the photosynthesis process has been overlooked in prior art. Consequently, many inventions have paid more attention to the volume of water and of the over diluted algal suspension being displaced than the actual available amount of photon per square meter available to that algal solution. This resulting low-efficiencies have lead to the necessity of oversizing algae farming facilities and consequently to high costs in investment, operations and energy.

### SUMMARY OF THE INVENTION

One object of the present invention is to provide a photobioreactor comprising at least one translucent flexible sheet shapable by a support assembly forming thereby an elongated channel adapted for biomass production therewithin.

Another object of the present invention is to provide a photobioreactor comprising at least one translucent flexible memory sheet shapable to form an elongated channel adapted to be mountable on a support assembly for biomass production therewithin.

Another object of the present invention is to provide a kit for making a photobioreactor, the kit comprising:
a support assembly; and
at least one translucent flexible sheet shapable by the support assembly forming
thereby an elongated channel adapted for biomass production therewithin.

Another object of the present invention is to provide a kit for making a floatable photobioreactor, the kit comprising;
a floating assembly; and
at least one translucent flexible memory sheet shapable to form an elongated channel, the elongated channel being mountable on the floating assembly forming thereby a floatable elongated channel adapted for biomass production therewithin.

### BRIEF DESCRIPTION OF THE DRAWINGS

These and other objects and advantages of the invention will become apparent upon reading the detailed description and upon referring to the drawings in which:

FIG. 1 is a perspective side view of the photobioreactor according to an embodiment of the present invention shaped for biomass production therewithin;

FIG. 2 is a perspective side view of the photobioreactor according to an embodiment of the present invention with a translucent cover;

FIG. 3 is a perspective front view of the photobioreactor according to an embodiment of the present invention with a translucent cover;

FIG. 4 is a perspective top view of the photobioreactor according to an embodiment of the present invention shaped by a bracket;

FIG. 5 is a perspective top view of the photobioreactor according to an embodiment of the present invention connected to a second photobioreactor;

FIG. 6 is a scheme top view of a sleeve according to an embodiment of the present invention with a gas sparger tube;

FIG. 7 is a perspective front view of the photobioreactor according to an embodiment of the present invention with a H-type extruded profile;

FIG. 8 is a perspective front view of the photobioreactor according to an embodiment of the present invention supported by a floating assembly;

FIG. 9 is a front view of the photobioreactor according to an embodiment of the present invention wrapped with a second translucent flexible sheet elevated by a frame;

FIG. 10 is a front view of the photobioreactor according to an embodiment of the present invention elevated by a frame and having an L-shape;

FIG. 11 is a perspective side view of the photobioreactor according to an embodiment of the present invention elevated by a shape-sustaining support;

FIG. 12 is a perspective side view of a shape-sustaining support according to an embodiment of the present invention;

FIG. 13 is a perspective top view of a water tank according to an embodiment of the present invention with a mixing system;

FIG. 14 is a perspective top view of a water tank according to an embodiment of the present invention with an external linear gas mixer device;

FIG. 15 is a perspective top view the evaporative water cooling system according to an embodiment of the present invention with an elevated wind turbine ventilator;

FIG. 16 is a perspective side view of the photobioreactor according to an embodiment of the present invention with a transparent electrochromatic panel

FIG. 17 is a perspective side view of the photobioreactor according to an embodiment of the present invention, supported by a floating assembly with a translucent cover;

FIG. 18 is a perspective side view of the photobioreactor according to an embodiment of the present invention, supported by a floating assembly; and

FIG. 19 is a perspective front view of the photobioreacor according to an embodiment of the present invention, suspended to trusses of a greenhouse.

### DETAILED DESCRIPTION OF PREFERRED EMBODIMENTS OF THE INVENTION

The present invention provides a photobioreactor made of a translucent flexible sheet or of a translucent flexible memory sheet that is shapable and rollable. The photobioreactor is thus easy to install and to transport at low cost. Further, the photobioreactor combines the control of microalgae culture typical to photobioreactor and the scability provided by pounds. The photobioreactor further maximizes exposure to sunlight with a high surface-to-volume ratio, minimizing water leakage and is rapid to assemble.

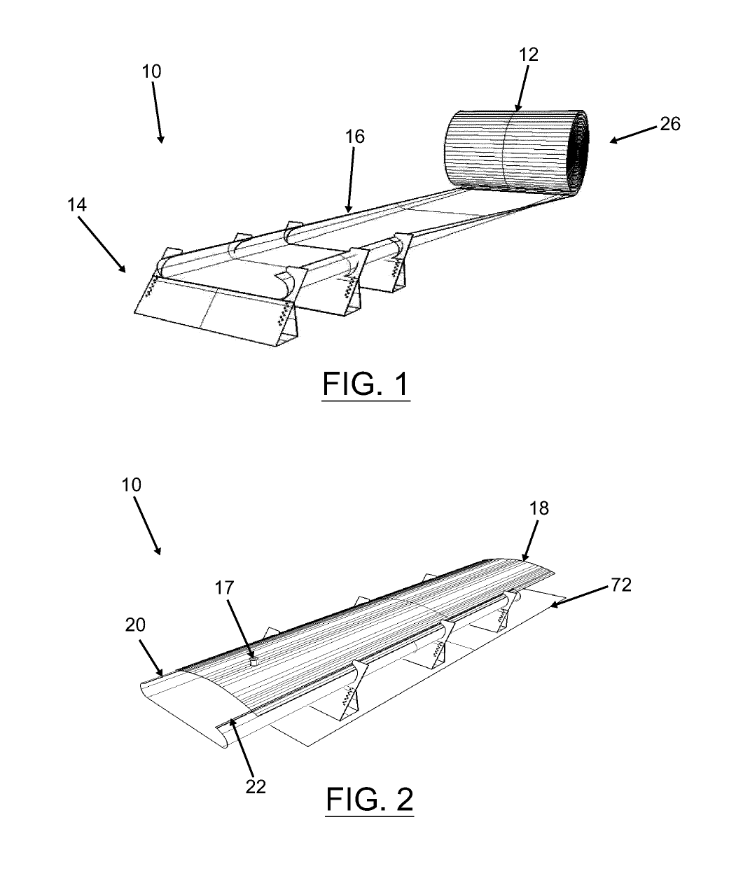

Referring to FIGS. 1 and 2, there is shown a photobioreactor 10 comprising at least one translucent flexible sheet 12. The translucent flexible sheet 12 is shaped by the support assembly 14 to form an elongated channel 16 adapted for biomass production therein.

In another embodiment, the photobioreactor 10 comprises at least one translucent flexible memory sheet which is shapable during manufacturing. In this embodiment, the translucent flexible memory sheet can be shaped by any known means to form an elongated channel such as by hands or by a support or may hold its shape by nature of shape memory provided during manufacturing. Thus in this embodiment, the shaped channel is adapted to be mounted on the support assembly 14.

As the translucent flexible sheet 12 and the translucent flexible memory sheet are flexible, they can be bent and/or rolled and can be provided in a compact roll reducing thereby transport, storage and installation costs of the photobioreactor.

In one embodiment, the photobioreactor comprises a translucent cover 18 attachable to opposite longitudinal edges 20 and 22 of the elongated channel 16. The translucent cover 18 can thus close a top portion of the elongated channel 16. The translucent cover 18 is attachable to the opposite longitudinal edges 20 and 22 by any known means such as but not limited to hooks or pressed between opposite longitudinal edges 20 and 22 and respectively upper portions of support assembly 14. The translucent cover 18 can be removed by being rolled or wrapped around a rotating horizontal axle from a trolley that is moved along and above the elongated channel 16. The removal of the translucent cover 18 may be automated. The translucent cover may comprise a porthole 17 for removal of gases or for introduction of elements into the bioreactor 10.

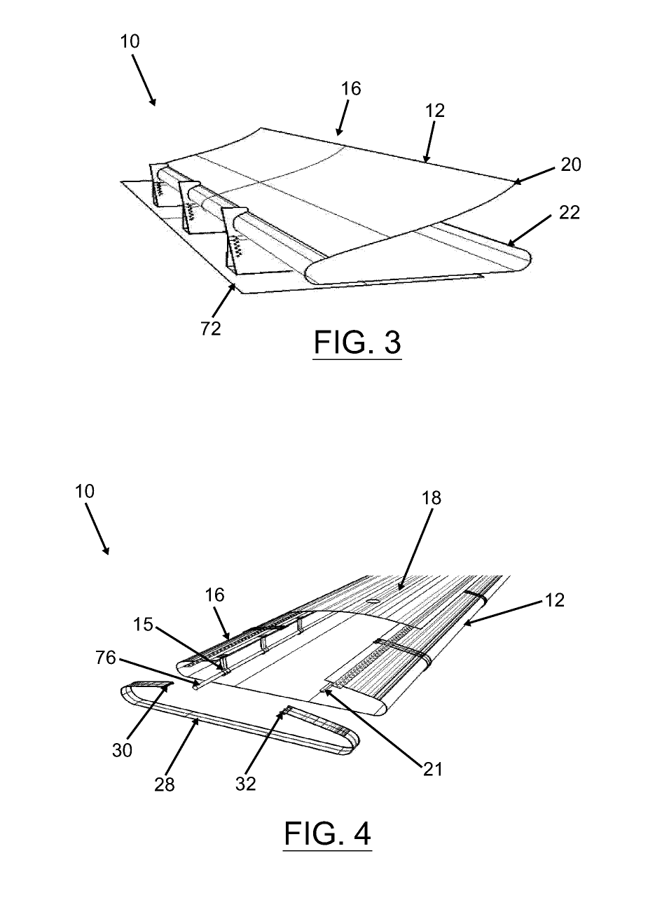

As shown at FIGS. 3, 7 and 8, a top portion of the elongated channel 16 can also be closed by attaching opposite longitudinal edges 20 and 22 to one another. In one embodiment, two translucent flexible sheets 12 or two translucent flexible memory sheets are longitudinally attached to one another using tape or any known chemical creating thereby a longer or a wider translucent flexible sheet 12 or a longer or wider translucent flexible memory sheet. In one embodiment, the opposite longitudinal edges 20 and 22 are attachable to one another using known means such as but not limited to hooks and loops, H-type extruded profile 24 or by pressing the opposite longitudinal edges 20 and 22 and nearest upper portion of support assembly 14.

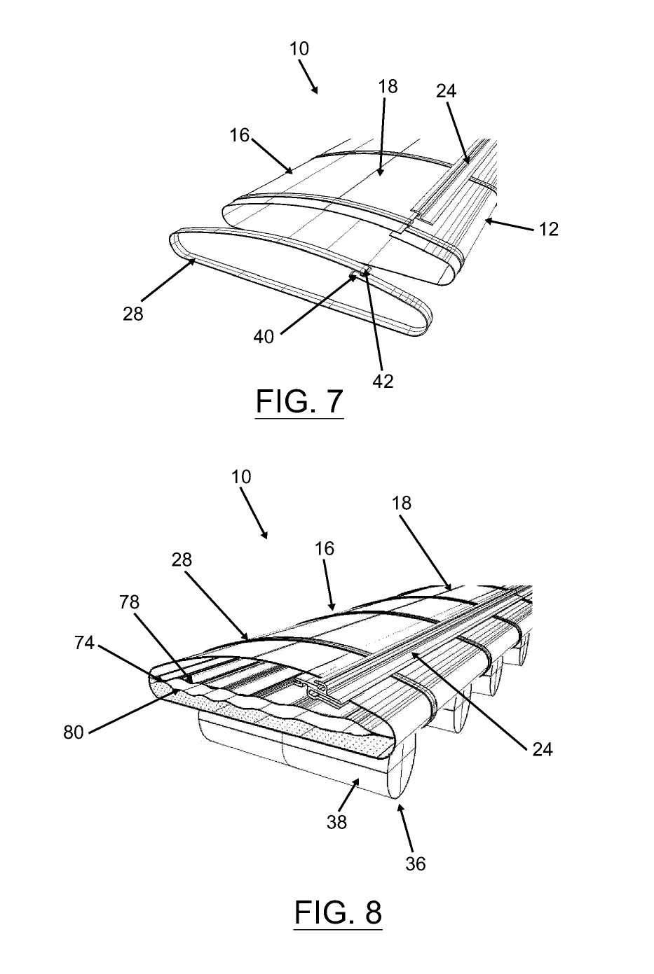

In one embodiment, the support assembly 14 may comprise a plurality of brackets 28 as shown at FIG. 4. The brackets 28 are disposable along the length of the translucent flexible sheet 12 or of the translucent flexible memory sheet shaping thereby said sheet. The brackets 28 comprise opposite ends 30 and 32 attachable to opposite longitudinal edges 20 and 22 of the translucent flexible sheet 12 or of the translucent flexible memory sheet forming or shaping thereby the elongated channel 16. In one embodiment, the opposite ends 30 and 32 of the brackets 28 are attachable to opposite longitudinal edges 20 and 22 with hooks 40 and 42 as shown at FIG. 7. The brackets 28 can have a C-shape or a L-shape forming thus a C-shaped or a L-shaped elongated channel 16 as shown at FIGS. 4 and 10. L-shaped brackets allow the formation of a water pocket 34 increasing residence time of mixing gases with water within the photobioreactor 10.

In another embodiment, the photobioreactor 10 can be positioned over a liquid surface. The photobioreactor 10 can float directly on the liquid surface or can comprise a floating assembly 36 which is mountable on and along the length of the elongated channel 16 as shown at FIG. 8. The floating assembly 36 allows the photobioreactor 10 to float on a surface such as a water channel, a dysfunctional raceway-type pond, a polluted water surface, a lake, a water reservoir, a swampy land where isolating the content of the photobioreactor 10 from a negative environment is required and vice-versa. The floating assembly 36 can comprise buoys 38 that may be pumped with a fluid or deflated to adjust height of the elongated channel 16. Rising or lowering the photobioreactor 10 may help discharge some of the semi-liquid content present within the photobioreactor 10 and may also be used to generate waves or vibrations creating thereby agitation of the liquid content within the photobioreactor 10. In one embodiment, the floating assembly 36 can comprise foil bubble-back insulation-type reflective film positioned underneath the photobioreactor 10 slightly above the liquid surface.

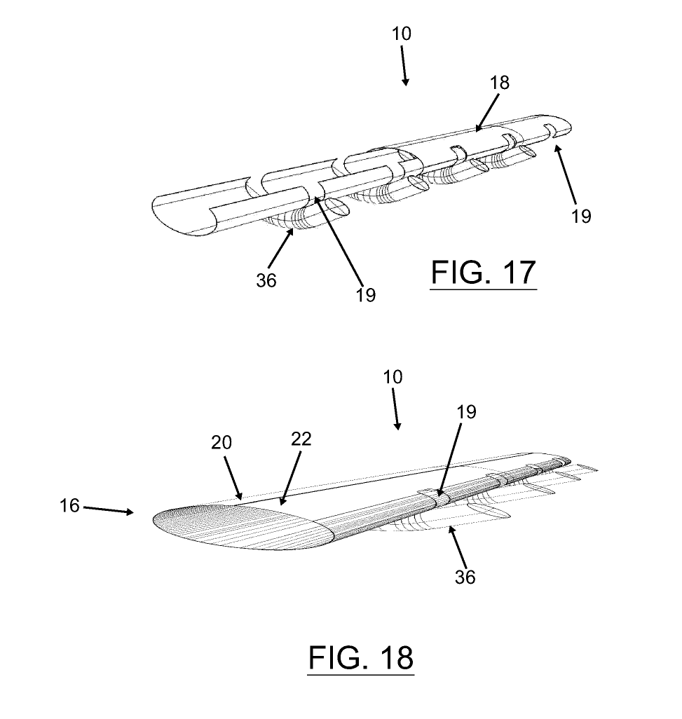

In one embodiment, the elongated channel 16 further comprises side-openings 19 which can be positioned at equal distance from each other on opposite sides of the translucent flexible sheet 12 as shown in FIG. 17. The floating assembly 36 can be adapted to engage the side-openings 19 supporting thereby the elongated channel 16 on a liquid surface. The floating assembly 36 can bend and twist with the movement of waves. A translucent cover 18 can have the same shape and size of the elongated channel 16 and can cooperate in a complementary manner with the elongated channel 16. The cover 18 can also have side-openings 19 located substantially at the same location as the one in the elongated channel 16. As shown at FIG. 18, the elongated channel 16 can comprise side-openings 19 when a top portion of the elongated channel is closed by attaching opposite longitudinal edges 20 and 22 to one another. To further provide a tight sealing between said opposite longitudinal edges 20 and 22, a long extruded plastic profile, such as an H-type extruded profile 24 may be provided to seal opposite edges of said translucent flexible sheet 12.

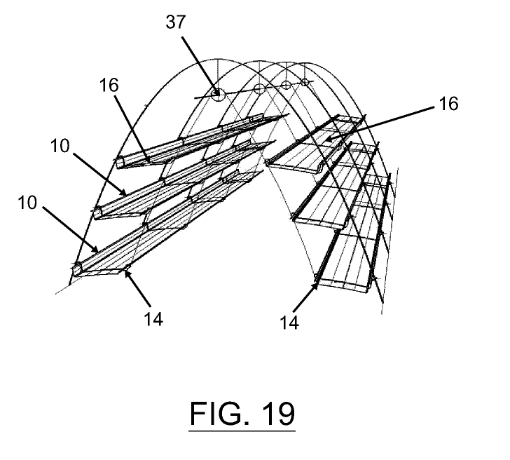

As shown at FIG. 19, multiple photobioreactors 10 can be suspended to trusses of a greenhouse. Weights and forces on trusses can be counterbalanced by an arrangement of pulleys and cables 37. Furthermore, because of balanced forces, the creation of waves along two cooperating elongated channels 16 requires only a small energy to rotate off-centered pulleys that connect cables on each side of two related support assembly 14.

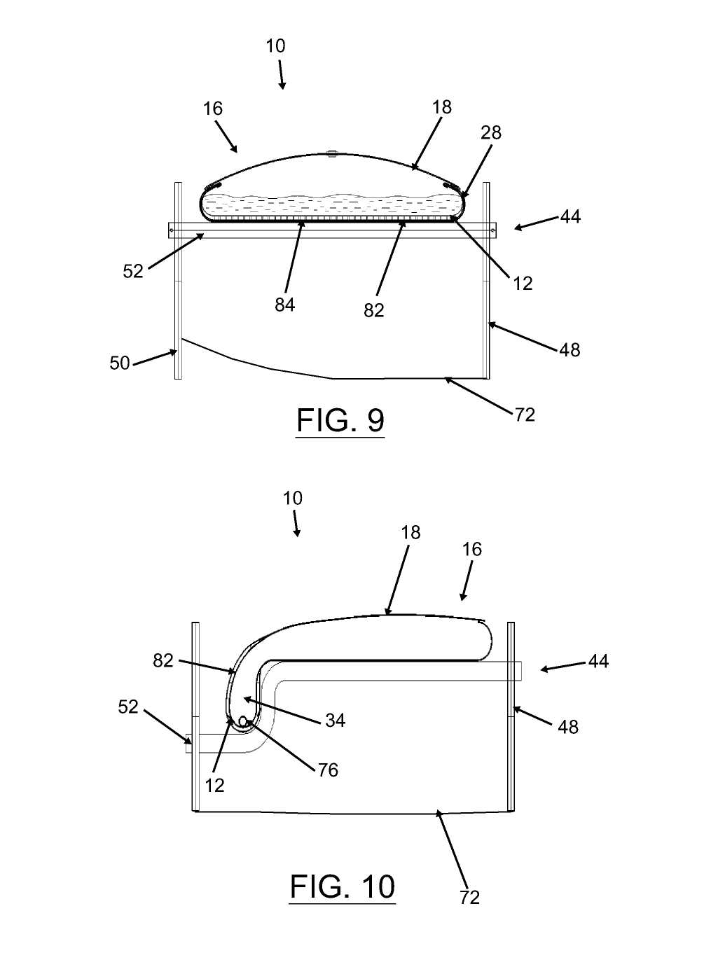

In another embodiment, the support assembly 14 can further comprise a plurality of frames 44, each frame 44 are adapted to elevate the elongated channel 16 above ground as shown at FIGS. 9 and 10. In one embodiment, the frames 44 are a scaffold-type frame. The elevated photobiorector 10 can be exposed to sunlight from all directions, including from underside. Each frame 44 can comprise a pair of vertical poles 48 and 50 and a horizontal pole 52 attachable to each vertical pole. The horizontal pole 52 may be affixed to the vertical pole 48 and 50 at a desirable height. The shape of the horizontal pole 52 can be adapted according to the shape of the elongated channel 16 as shown at FIGS. 9 and 10. When the elongated channel 16 is provided with a water pocket 34, the horizontal pole 52 can have a L-shape or a S-shape as shown at FIG. 10. The frames 44 can also be stacked above each in order to obtain multiple photobioreactors 10 staged above each other to reduce the bioreactor footprint.

The support assembly 14 can further comprise a plurality of shape-sustaining supports 54 as shown at FIGS. 11, 12 and 16. Each shape-sustaining support 54 is mountable on and along the length of the translucent flexible sheet 12 or the translucent flexible memory sheet to be shaped forming thereby the elongated channel 16. In one embodiment, each shape-sustaining support 54 comprises a base 56 and a pair of projections 58 and 60 extending upwardly from the base 56 defining a cavity 62 therebetween. The cavity 62 is adapted to receive the translucent flexible sheet 12 or the translucent flexible memory sheet such that each opposite longitudinal edges 20 and 22 of the translucent flexible sheet 12 or the translucent flexible memory sheet engages the projections 58 and 60. The shape of the cavity 62 defines the shape of the elongated channel 16. The base 56 may further comprise a recess 64 in communication with the cavity 62 such that the translucent flexible sheet or the translucent flexible memory sheet is further engagable within the recess 64. The recess 64 allows the formation of a water pocket 34 as previously described increasing gas residence time. The base 56 may comprise a plurality of recesses forming thus a T-shape, a M-shape, U-shape or a W-shape. In one embodiment, the recess 64 has an oblique shape. In another embodiment, the recess is O-shape wherein a generally flat C-shape configuration evolves into an O-shape or a funnel-shape.

In another embodiment, the width of the cavity 62 and the shape of the recess 64 may vary from one shape-sustaining support to another. Thus the width and the depth of the elongated channel 16 and the shape of the water pocket 34 may vary along the length of the elongated channel 16. In a first example, the shape of the elongated channel 16 may vary from a generally oval-shape channel into a funnel-shape channel, thus gradually funneling algal flow into a harvester system (not shown) for dewatering and extraction of algal oil. In another embodiment, a T-shape elongated channel 16 may gradually take on a different shape such as M-shape and finish into a cylindrical-shape or an inclined-shape elongated channel. Each of these shapes has their own merits. For example, an inclined-shape channel enables to have deeper water on one side of the channel which results into an increase of residence time during mixing of gases with water. Often microalgae inoculation is done in a closed photobioreactor prior to transferring the resulting culture into an opened or closed photobioreactor for mass culture. The shape of the photobioreactor 10 can be adapted to enhance both the control existing in photobioreactor and the scalability of opened or closed ponds, without facing challenges of connectivity between the two systems.

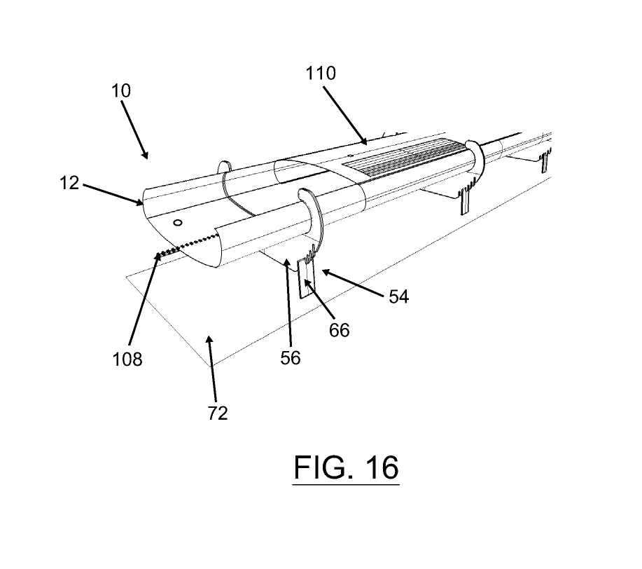

In another embodiment, the height of the base 56 is adjustable by any known means. For example, each base 56 may comprise a leg 66 mountable thereon further elevating the elongated channel 16 above the ground. The leg 66 can be slidebly mountable on the base 56 using rails as shown at FIG. 11 or can be insertable in grooves provided in the base 56 as shown at FIG. 16.

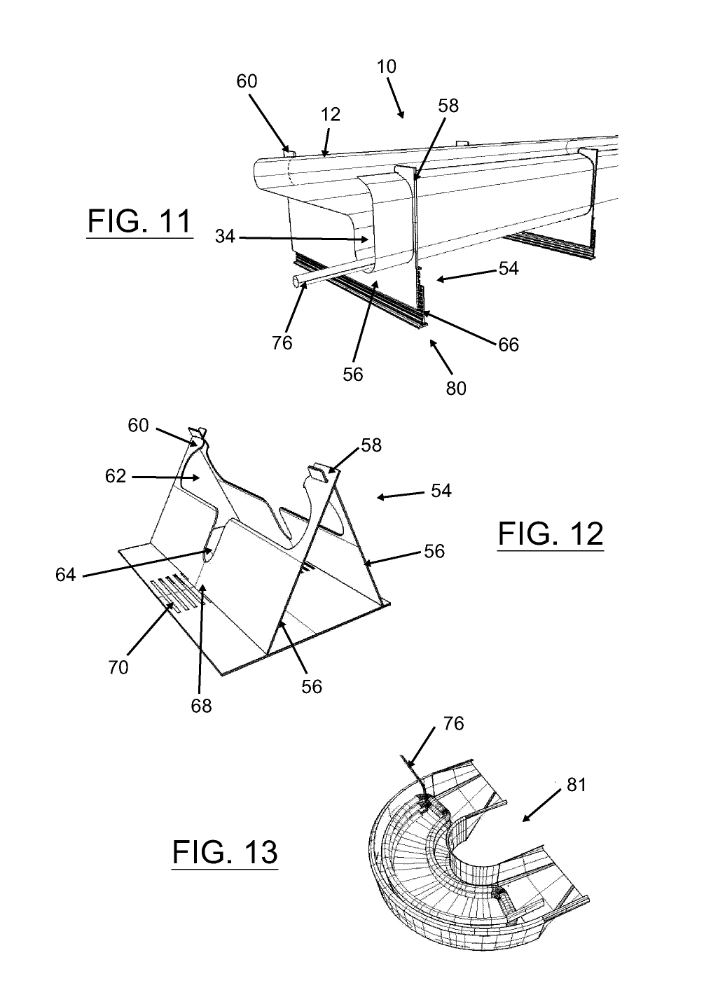

In another embodiment, the base 56 comprises a height-adjustable delta-shape as shown at FIGS. 1 and 12. In one embodiment, the pair of projections 58 and 60 of a first shape-sustaining support 54 is rotatably attachable to a pair of projections 58 and 60 of a second shape-sustaining support 54. In this embodiment, the height of each base 56 can be adjusted by any known means. For instance, the base 56 can be provided with a tongue 68 which can engage with at least one groove 70 located adjacent the tongue 68 securing thereby the base. As shown at FIG. 12, the tongue 68 can extend downwardly from the base 56 and can engage with a groove 70 provided underneath the elongated channel 16. In another embodiment, the tongue 68 can further be attached to the groove 70 via screws, nylon ties or any suitable fastening means. By providing a plurality of grooves adjacent the tongue, the height of the base 56 can be adjusted. The height of the first shape-sustaining support can thus be adjusted by rotatably changing the angle between the first and second shape-sustaining support.

The first shape-sustaining support and the second shape-sustaining support transfer weight of the elevated photobioreactor 10 across its width to the ground. The distribution of weight allows the base 56 to use low-cost construction materials such as wood, plastic-lumber, fiberglass, fiber-cement, clay, magnesium oxide, gypsum, metal plate or a combination thereof.

The height of the photobioreactor 10 can substantially be maintained constant along its length by adjusting the height of the frame 44 or by adjusting the height of the shape-sustaining support 54. Furthermore, the height of the photobioreactor 10 can also vary along its length by adjusting the height of the frame 44 or by adjusting the height of the shape-sustaining support 54. This becomes advantageous to create a cascade where algal solution can flow from higher level of the elongated channel 16 into gradually a lower level of the elongated channel 16, thus reducing the need for pumps.

The photobioreactor 10 can also comprise a reflective material 72 located adjacent the elongated channel 16 as shown at FIGS. 9 and 10. The reflective material 72 enhances the beneficiary effect of the photosynthesis process. In one embodiment, the reflective material 72 is located underneath the elongated channel 16 and can be laid over a ground surface. The reflective material 72 can be oriented with an angle or a curve or may be laid flat over the ground. The reflective material can comprise reflective paint, reflective film, reflective mineral, or foil bubble-back insulation-type reflective film. When the photobioreactor 10 comprises a floating assembly, the reflective material 72 can be a floating reflective material. In another embodiment, the reflective material 72 can be provided on the frame 44 or on the shape-sustaining support 54.

The photobioreactor 10 may further comprise a translucent sleeve 74 insertable into the elongated channel 16 for biomass production therewithin as shown at FIGS. 6 and 8. The sleeve 74 reduces the cleaning and the maintenance needs of the photobioreactor 10. Liquid such as water may be circulated around the sleeve 74 to control the temperature of the sleeve content. The sleeve 74 may be sterilized by any known means such as gamma ray or by containing antibacterial additives providing a sterile translucent environment for more sensitive microalgae strains. The sleeve 74 may also contain other additives that enhance algal growth such as UV and/or IR (Infra-Red) absorbing additives, dyes, nanoparticles or a combination thereof.

In another embodiment, as shown at FIG. 6, the sleeve 74 comprises an internal gas sparger tube 76 for delivery of air and carbon dioxide inside the sleeve 74. The gas sparger tube 76 can be made of a thin plastic disposable, recyclable or biodegradable material such as the one used for drip irrigation reducing cleaning and maintenance problems associated with photobioreactor. The gas sparger tube 76 may also be made of a gas-permeable material such as, but not limited to, rubber particles. The gas sparger tube 76 can be made of the same material as sleeve 74 by tucking a small part of sleeve 74 into its own edge, thus forming sparger tube 76 at the same time as sleeve 74 is being shaped. Similar to the shaping of a gusseted tubing which has a triangular shaped pleat on one side of a layflat tube, there is provided a lay flat tube or sleeve 74 that includes a triangular shaped pleat first punched with pin holes 75 and then, having the base of the triangle sealed so as to create an internal gas sparger tube 76 within sleeve 74.

In one embodiment, the photobioreactor 10 may comprise a dewatering system. To dewater the biomass, the sleeve 74 may be provided with an upper translucent film 78 and a bottom osmosis membrane 80. The sleeve 74 can be partially filled with a liquid such as fresh water and a biomass suspension. The sleeve 74 can be adapted to float within the photobioreactor 10 over a fluid of higher solute concentration than its own fluid content such as sea water. It is known that any liquid of lower solute concentration flows through an osmosis membrane to a liquid of higher solute concentration to seek equilibrium. This flow effect causes dehydration of biomass.

In another embodiment, dewatering of the biomass may be achieved by providing the sleeve 74 made entirely of an osmosis membrane partially filled with salt water. Water content in diluted biomass present in the photobioreactor 10 permeates through the osmosis membrane of sleeve 74 and flows towards the higher solute concentration present within the sleeve 74 causing dehydration of biomass.

To enhance biomass growth, the translucent flexible sheet, the translucent flexible memory sheet or the translucent sleeve may comprise antibacterial additives, anti-rotifier additives, ultra-violet absorbent, infra-red absorbent, ultra-violet and infra-red blocker film, additives or film absorbing photo inhibitive wavelengths, spectral shifting dyes, absorbents for all sunlight wavelengths except wavelengths between 400 nm and 700 nm absorbents for all sunlight wavelengths except wavelengths between 660 to 700 nm or a combination thereof.

In another embodiment, the photobioreactor 10 may further comprise a temperature controlling system. A second translucent flexible sheet 82 or a second translucent flexible memory sheet shapable by the support assembly 14 may be wrapped around the elongated channel 16 as shown at FIGS. 9 and 10. Spacers 84 may further be positioned along the length of the elongated channel and between the second translucent flexible sheet 82 and the first translucent flexible sheet creating therebetween a space similar to a water jacket. Liquid such as water may be circulated in this water jacket between the second translucent flexible sheet 82 and the first translucent flexible sheet 12 for controlling temperature within the photobioreactor 10.

In one embodiment, the translucent flexible sheet 12, the translucent flexible memory sheet, the second translucent flexible sheet 82, the second translucent flexible memory sheet or the translucent cover 18 is made of a material such as fiber reinforced plastic, low density polyethylene, high-density polyethylene, hard acrylic, polyvinyl chloride, polycarbonate, composite plastic, ethylene vinyl acetate, fiberglass and a combination thereof. Fiberglass offers advantages such as durability and ease of repair and maintenance. A fiberglass sheet typically may last as long as 25 years or more making return on investment substantially affordable.

The translucent flexible sheet 12, the translucent flexible memory sheet, the second translucent flexible sheet 82, the second translucent flexible memory sheet or the translucent cover 18 may be about 0.5 mm to 1.2 mm thick, about 3 m to 50 m long and about 0.5 m to 2.5 m wide.

In another embodiment, the translucent flexible sheet 12, the translucent flexible memory sheet, the second translucent flexible sheet 82, the second translucent flexible memory sheet or the translucent cover 18 may further include attached thereto or embedded therein a biomass growth monitor assembly, a biomass growth detector assembly and/or a biomass growth promoter assembly. These assemblies may comprise the following components: flexible wire, sensor, light emitting diode, optical sensor, Bluetooth short-range connection, photovoltaic cell, microplate reader, batterie, piezo-electric vibrator, thermotropic crystal, liquid crystal, suspended particle display, electrochromic film, reflective hydride, heating element, heating tape, wire to generate electromagnetic field, electrode and a combination thereof. To enhance biomass productivity, the electronically-connected devices mentioned above, may be electronically pulsated so as to manipulate intensity and frequency of light sources, to flash light, to generate magnetic waves or to generate electrical pulses that enhance the oil extraction process.

Further, as shown at FIG. 4, various devices may be attached along the longitudinal edges 20 and/or 22 of the translucent flexible sheet 12 or to the H-type extruded profile 24 such as hangers 15 for holding the gas sparger tube 76, LED tapes 21 or LED ropes, instruments and other devices that promote, detect or monitor biomass growth as described above.

Agitation of the biomass within the photobioreactor 10 can be achieved by any known means such as a wave generation system, pump or water wheel. When the production of a sterile cultivation of the biomass is required, the agitation equipment is configured to maintain a degree of air-tightness that prevents air contamination from outside. The suspended algal solution within the translucent sleeve 74 is agitated by one or multiple wave generators which can comprise bellows that inflate at controlled time by lifting at a time interval the sleeve 74 creating thus a wave moving along the length of the photobioreactor 10. Once the wave has reached its destination, the sleeve 74 is lowered and lifted again to generate the following wave and the cycle is repeated. In one embodiment, a wave generation system is connectable to one end of the photobioreactor 10. In one embodiment, a wave generator connectable to one end of the photobioreactor 10 lifts angularly a volume of water and empties it to flow in the elongated channel 16.

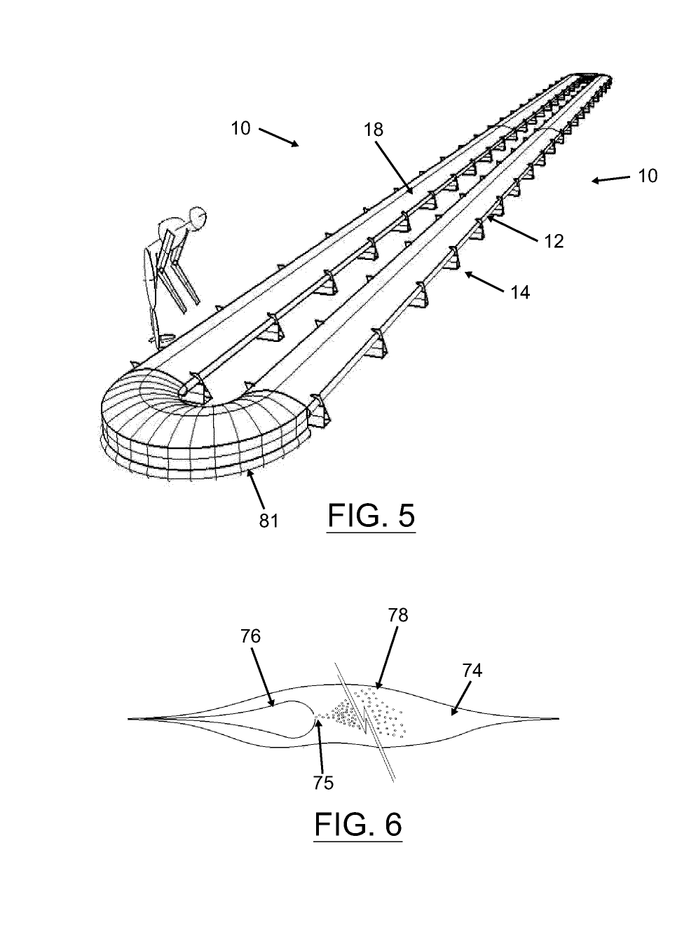

Referring to FIG. 5, there is shown two photobioreactors 10 in fluid communication with each other. In one embodiment, the photobioreactors 10 can be in fluid communication via water tank 81 or pipes mountable at each end of the photobioreactors 10. The water tank 81 can have a U-shape as shown at FIG. 13. In another embodiment, at least one end of the photobioreactor 10 is closed using known means such as a bulkhead. The bulkhead can have a flat plate-shaped body configured with substantially a similar cross-sectional dimension than the elongated channel 16 surrounded by a soft seal affixed to the bulkhead contour. The bulkhead may also be placed anywhere and at any time inside the photobioreactor 10 to close or isolate a section thereof. Isolation of a partial section can occur both with (over sleeve 74) or without sleeve 74 being present. Extension of length of a photobioreactor 10 can be achieved by adhering overlapping ends of two adjacent translucent flexible sheets 12. In one embodiment, the shape-sustaining support 54 or the frame 44 is located at a position where the two elongated channels 16 are joined together.

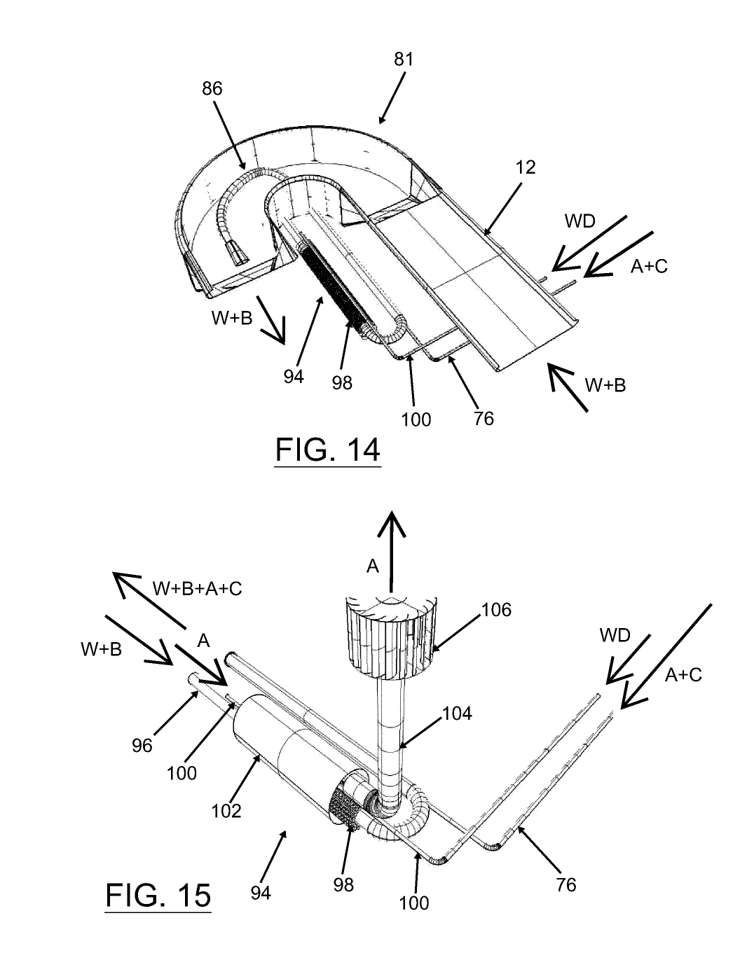

As shown in FIGS. 14 and 15, to maintain the temperature of photobioreactor 10 within a range of about 15° Celcius to a about 30° Celcius, an evaporative water cooling system 94 comprising an elongated heat pipe 96 such as a metal pipe, contains circulating water in fluid communication with the bottom of the photobioreactor 10. The heat pipe 96 can be surrounded by a layer of a porous material 98 such as charcoal, expanded clay pebbles, evaporative wick, and porous materials or the like. The porous material 98 can be contained inside a wire meshing. A drip watering system 100 can be positioned above the porous material 98 and is continuously or automatically wetting the porous material 98. An elongated larger enclosure 102 can surround the wire meshing. One end of the air duct 104 can be partially inserted inside the elongated larger enclosure 102 and the other end can be attached to an elevated wind turbine ventilator 106. The elevated wind turbine ventilator 106 creates an air draft in the elongated larger enclosure 102 sucking air therethrough and causing evaporation of moisture present in the porous material 98 which in turn creates a cooling effect of the elongated heat pipe 96. When the evaporative water cooling system 94 operates in tandem with the elongated gas mixing device 86, a natural water circulation is created in the elongated heat pipe 96, increasing the efficiency of both the low-cost mixing system and the low-cost cooling systems.

Arrows in FIGS. 14 and 15 represent fluid flows. WD represents the flow direction of the drip watering system 100, A+C represents Air and Carbon dioxide flow in gas sparger tube 76, W+B represents flow of Water and Biomass. Similarly, W+B+A+C represents the flow of Water, Biomass mixed with Air and Carbon dioxide.

Referring to FIG. 16, there is shown electric devices 108 and 110 which can be embedded or attached to the translucent flexible sheet 12 or to the translucent memory sheet to enhance microalgae growth. The pulsating on and off electric device 110 has multiple benefits. For example, a bio-tuning effect occurs when the electronic device 110, for example a transparent electrochromatic panel, is pulsated, causing the intensification of algal growth under a flashing light effect. This is also a way to control the intensity of sunlight in hot climates. Further selected light spectrum can be generated to intensify algal growth.

In another embodiment, the present invention provides a kit for making a photobioreactor. The kit comprises the support assembly 14 and at least one translucent flexible sheet 12 shapable by the support assembly 14 forming thereby the elongated channel 16 adapted for biomass production therewithin. The kit may further comprise the above-mentioned elements.

In another embodiment, the present invention provides a kit for making a floatable photobioreactor. The kit comprises a floating assembly 36 and at least one translucent flexible memory sheet shapable to form an elongated channel. The elongated channel is mountable on the floating assembly 36 forming thereby a floatable elongated channel adapted for biomass production therewithin. The kit may further comprise the above-mentioned elements.

As the translucent flexible sheet or the translucent flexible memory sheet is made of flexible sheet such as fiberglass, the bending stress applied to shape them into an elongated channel is well tolerated by the flexible sheet. Consequently, the elongated channel may be spanned or elevated at a longer distance than a typical sheet. This translates into longer span than may be projected between load-bearing or support assembly than a typical sheet. This advantage reduces costs and makes commercial scale-up of biomass production more affordable.

Further, the photobioreactor of the present invention has the advantage of combining the scalability and cost-effectiveness offered by open ponds with the biomass growth control provided by photobioreactors such as providing a high surface-to-volume light exposure ratio and light exposure from different directions.

The scope of the claims should not be limited by the preferred embodiments set forth in the examples, but should be given the broadest interpretation consistent with the description as a whole.

1-38. (canceled)

39. A photobioreactor comprising at least one semi-rigid translucent flexible sheet provided with pre-shaped borders, said pre-shaped sheet forming an elongated channel adapted to be mountable on a support assembly for biomass production therewithin; said channel shape comprising a C-shape, an L-shape, a T-shape, an M-shape, an O-shape, a funnel-shape or a W-shape and said sheet reducible into a roll.

40. The photobioreactor of claim 39, further comprising a translucent cover attachable to opposite longitudinal edges of the elongated channel closing thereby a top portion of the elongated channel.

41. The photobioreactor of claim 39, wherein the support assembly comprises a plurality of height adjustable frames, each frame being adapted to elevate the elongated channel above ground.

42. The photobioreactor of claim 39, wherein the support assembly is made of a material selected from the group consisting of wood, plastic-lumber, fiberglass, fibrocement, clay, magnesium oxide, gypsum, metal and a combination thereof.

43. The photobioreactor of claim 39, further comprising a translucent flexible sleeve insertable into the elongated channel for biomass production therewithin.

44. The photobioreactor of claim 43, wherein the translucent sleeve comprises at least a flexible gas sparger tube formed from the sleeve, therewithin.

45. The photobioreactor of claim 44, wherein the translucent sleeve is made of a material selected from the group consisting of: plastic, film, osmosis membrane and a combination thereof.

46. The photobioreactor of claim 39, wherein the at least one the translucent flexible pre-shaped sheet or the translucent sleeve comprises antibacterial additive, anti-rotifer additive, ultra-violet absorbent, infra-red absorbent, ultra-violet and infra-red blocker film, additives or film absorbing photo inhibitive wavelengths or a combination thereof.

47. The photobioreactor of any claim 39, wherein the at least one the translucent flexible pre-shaped sheet, or the translucent cover is made of a material selected from the group consisting of: fiber reinforced plastic, low density polyethylene, high-density polyethylene, hard acrylic, polyvinyl chloride, polycarbonate, composite plastic, ethylene vinyl acetate, fiber glass and a combination thereof.

48. The photobioreactor of claim 47, wherein the at least one the translucent flexible pre-shaped sheet, or the translucent cover is about 0.5 mm to 2 mm thick, about 3 m to 100 m long and about 0.5 m to 2.5 m wide.

49. The photobioreactor of claim 39, further comprising a wave generation system connectable to one end of the elongated channel agitating thereby the biomass along the length of the channel.

50. The photobioreactor of claim 39, further comprising a biomass growth monitor assembly, a biomass growth detector assembly and electrical means for promoting biomass growth.

51. The photobioreactor of claim 50, wherein the biomass growth monitor assembly, the biomass growth detector assembly or electrical means for promoting biomass growth comprise components selected from the group consisting of: flexible wire, sensor, light emitting diode, optical sensor, Bluetooth short-range connection, photovoltaic cell, microplate reader, batteries, piezoelectric vibrator, thermotropic crystal, liquid crystal, suspended particle display, electrochromic film, reflective hydride, heating element, heating tape, wire to generate electromagnetic field, electrode and a combination thereof.

52. The photobioreactor of claim 39, wherein the photobioreactor is adapted to be suspended to greenhouse trusses.

53. A kit for making a photobioreactor, the kit comprising:
a translucent flexible sleeve and at least one translucent semi-rigid flexible sheet provided with pre-shaped borders; said sheet adapted to form an elongated channel; said sleeve insertable into the said flexible sheet; said kit adapted for biomass production therewithin; said channel shape comprising a C-shape, an L-shape, a T-shape, an M-shape, an O-shape, a funnel-shape or a W-shape and said sheet reducible into a roll.

54. A flexible photobioreactor system comprising:
a translucent flexible sleeve adapted for biomass growth; and
a gas sparger tube made of the same material as said sleeve, said sparger tube shaped by tucking a small part of the sleeve first punched with pin holes and then tucked into the sleeve own edge in a manner similar to the shaping of a gusseted lay flat tubing having a triangular shaped pleat, and finally sealed so as to create an internal gas sparger tube within the sleeve.
